(19) United States
(12) Patent Application Publication
(10) Pub. No.: US 2026/0216417 A1
(43) Pub. Date: Jul. 30, 2026

(54) Fluid Control Device For Negative Pressure Wound Therapy System

(71) Applicant: Solventum Intellectual Properties Company, Maplewood, MN (US)

(72) Inventors: Andrew J. MCGREGOR, San Antonio, TX (US); Christopher J. CARROLL, San Antonio, TX (US); Richard M. KAZALA, San Antonio, TX (US); Jonathan G. REHBEIN, San Antonio, TX (US); Javier GONZALEZ, San Antonio, TX (US)

(21) Appl. No.: 19/146,435

(22) PCT Filed: Jan. 9, 2024
(86) PCT No.: PCT/IB2024/050196

§ 371 Date: Jul. 8, 2025

Related U.S. Application Data
(60) Provisional application No. 63/440,960, filed on Jan. 25, 2023.

(51) Int. Cl. A61M 1/00; A61F 13/05

(52) U.S. Cl. CPC A61M 1/962; A61F 13/05; A61M 1/98

(57) ABSTRACT

The present disclosure provides a fluid control device for use with a negative pressure wound therapy system including a pump having an inlet and an outlet. The fluid control device includes a first valve, a second valve, and a canister assembly. The canister assembly includes a first canister including a first chamber and a first diaphragm dividing the first chamber into a first air chamber and a first exudate chamber. The canister assembly further includes a second canister including a second chamber and a second diaphragm dividing the second chamber into a second air chamber and a second exudate chamber. The first valve is configured to selectively fluidly communicate the inlet with one of the first air chamber and the second air chamber. The second valve is configured to selectively fluidly communicate the outlet with the other of the first air chamber and the second air chamber.

### CROSS-REFERENCE TO RELATED APPLICATIONS

This application is a U.S. National Stage Entry of PCT International Application No. PCT/IB2024/050196, filed Jan. 9, 2024, which claims the benefit of priority to U.S. Provisional Application No. 63/440,960, filed on Jan. 25, 2023, each of which are incorporated herein by reference in their entirety.

### TECHNICAL FIELD

The present disclosure generally relates to a negative pressure wound therapy (NPWT) system and more particularly to a flow control device for use with the NPWT system.

### BACKGROUND

Caring for wounds is important in a healing process. Wounds generally produce fluids, e.g., an exudate, such as, slough, necrotic tissue, or microbial load (e.g., bacteria and biofilms). If not properly addressed, the exudate can lead to infection or maceration of a wound site.

Negative pressure wound therapy (NPWT) systems are embodied as sealed wound-care systems particularly indicated for wounds, such as, chronic persistent wounds and/or complicated wounds. Specifically, for promoting wound healing, a pressure that is reduced relative to the surroundings (commonly referred to as “negative pressure”) is applied to a wound site. The negative pressure causes mechanical contraction of the wound and removal of the exudate from the wound site, thus promoting formation of granulation tissues and accelerating wound healing. The NPWT system typically includes a therapy unit that is in fluid communication with the wound site.

The exudate removed from the wound site is generally collected in a rigid canister for disposal or analysis. Typically, to manage low levels of the exudate, the use of rigid canisters to store the exudate may increase patient discomfort as they may make the NPWT system bulky to handle and may affect a mobility of patient. Further, the rigid canisters may also impact a portability of the NPWT system. In some examples, conventional wound dressings may include a superabsorbent to absorb the exudate. When such wound dressings get saturated with the exudate, the wound dressings may adversely affect a transfer of the negative pressure to the wound site as the exudate may block pathways for negative pressure flow, which may lead to loss of therapy. In other cases, the exudate collected in the wound dressing itself may present a risk of maceration if the absorbent is fully saturated. Further, some wound dressings may not completely isolate components of the NPWT system, such as, the pump from the exudate, which may allow ingress of the exudate into the components of the NPWT system, which is not desirable.

### SUMMARY

Generally, the present disclosure relates to a fluid control device and a negative pressure wound therapy system including the fluid control device.

In a first aspect, the present disclosure provides a fluid control device for use with a negative pressure wound therapy (NPWT) system having a wound dressing, a pump, and an exudate canister configured to receive wound exudate removed from the wound site. The fluid control device includes a first valve disposed in fluid communication with an inlet of the pump. The fluid control device further includes a second valve disposed in fluid communication with an outlet of the pump. The fluid control device further includes a canister assembly. The canister assembly includes a first canister including a first chamber and a first diaphragm disposed in the first chamber and dividing the first chamber into a first air chamber and a first exudate chamber. The first diaphragm further fluidly isolates the first air chamber from the first exudate chamber. The first air chamber is disposed in fluid communication with each of the first valve and the second valve. The first exudate chamber is configured to be in selective fluid communication with the wound dressing and the exudate canister. The canister assembly further includes a second canister fluidly isolated from the first canister. The second canister includes a second chamber and a second diaphragm disposed in the second chamber and dividing the second chamber into a second air chamber and a second exudate chamber. The second diaphragm further fluidly isolates the second air chamber from the second exudate chamber. The second air chamber is disposed in fluid communication with each of the first valve and the second valve. The second exudate chamber is configured to be in selective fluid communication with the wound dressing and the exudate canister. The first valve is configured to selectively fluidly communicate the inlet with one of the first air chamber of the first canister and the second air chamber of the second canister. The second valve is configured to selectively fluidly communicate the outlet with the other of the first air chamber of the first canister and the second air chamber of the second canister.

In a second aspect, the present disclosure provides a negative pressure wound therapy (NPWT) system for treatment of a wound site. The NPWT system includes a wound dressing disposed at the wound site. The NPWT system further includes an exudate canister configured to receive wound exudate removed from the wound site. The NPWT system further includes a pump including an inlet and an outlet. The pump is configured to provide a negative pressure at the inlet and a positive pressure at the outlet. The NPWT system further includes a first valve disposed in fluid communication with the inlet of the pump. The NPWT system further includes a second valve disposed in fluid communication with the outlet of the pump. The NPWT system further includes a canister assembly. The canister assembly includes a first canister including a first chamber and a first diaphragm disposed in the first chamber and dividing the first chamber into a first air chamber and a first exudate chamber. The first diaphragm further fluidly isolates the first air chamber from the first exudate chamber. The first air chamber is disposed in fluid communication with each of the first valve and the second valve. The first exudate chamber is configured to be in selective fluid communication with the wound dressing and the exudate canister. The canister assembly further includes a second canister fluidly isolated from the first canister. The second canister includes a second chamber and a second diaphragm disposed in the second chamber and dividing the second chamber into a second air chamber and a second exudate chamber. The second diaphragm further fluidly isolates the second air chamber from the second exudate chamber. The second air chamber is disposed in fluid communication with each of the first valve and the second valve. The second exudate chamber is configured to be in selective fluid communication with the wound dressing and the exudate canister. The first valve is configured to selectively fluidly communicate the inlet with one of the first air chamber of the first canister and the second air chamber of the second canister. The second valve is configured to selectively fluidly communicate the outlet with the other of the first air chamber of the first canister and the second air chamber of the second canister.

In a third aspect, a method for controlling fluid flow in the NPWT system of the second aspect in provided. The method includes controlling the first valve and the second valve, such that the first valve fluidly communicates the inlet with one of the first air chamber of the first canister and the second air chamber of the second canister, and the second valve fluidly communicates the outlet with the other of the first air chamber of the first canister and the second air chamber of the second canister.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic view illustrating a negative pressure wound therapy (NPWT) system for treatment of a wound site, according to an embodiment of the present disclosure;

FIG. 2 illustrates a schematic block diagram of a fluid control device of the NPWT system of FIG. 1, according to an embodiment of the present disclosure;

FIG. 3 illustrates a perspective top view of a canister assembly associated with the fluid control device of FIG. 1, according to an embodiment of the present disclosure;

FIG. 4A illustrates a sectional side view of a first canister of the canister assembly when the NPWT system is in a first state, according to an embodiment of the present disclosure;

FIG. 4B illustrates a sectional side view of a second canister of the canister assembly when the NPWT system is in the first state, according to an embodiment of the present disclosure;

FIG. 4C illustrates a sectional bottom view of the canister assembly of FIG. 3, when the NPWT system 100 is in the first state, according to an embodiment of the present disclosure;

FIG. 5A illustrates a sectional side view of the first canister of the canister assembly when the NPWT system is in a second state, according to an embodiment of the present disclosure;

FIG. 5B illustrates a sectional side view of the second canister of the canister assembly when the NPWT system is in the second state, according to an embodiment of the present disclosure;

FIG. 5C illustrates a sectional bottom view of the canister assembly of FIG. 3 when the NPWT system is in the second state, according to an embodiment of the present disclosure;

FIG. 6 is a schematic view illustrating the NPWT system for treatment of the wound site, according to another embodiment of the present disclosure; and

FIG. 7 illustrates a flowchart for a method for controlling fluid flow in the NPWT system of FIG. 1, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As used herein, all numbers should be considered modified by the term “about”. As used herein, “a,” “an,” “the,” “at least one,” and “one or more” are used interchangeably.

The term “about”, unless otherwise specifically defined, means to a high degree of approximation (e.g., within +/−5% for quantifiable properties) but again without requiring absolute precision or a perfect match.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

The term “coupled”, or “connected” may include direct physical connections between two or more components, or indirect physical connections between two or more components that are connected together by one or more additional components. For example, a first component may be coupled to a second component by being directly connected together or by being connected by a third component.

As used herein, the term “configured to” and like is at least as restrictive as the term “adapted to” and requires actual design intention to perform the specified function rather than mere physical capability of performing such a function.

As used herein, the terms “layer,” “sheet,” and “dressing,” or variations thereof, are used to describe an article having a thickness that is small relative to its length and width.

As used herein, the term “negative pressure” broadly refers to a pressure lower than a local pressure in a local environment outside of a sealed treatment environment provided by a dressing. In many cases, the local ambient pressure can also be the atmospheric pressure at which a wound site is located. Alternatively, the pressure can be less than a hydrostatic pressure associated with a tissue at the wound site.

As used herein, the term “positive pressure” broadly refers to a pressure higher than a local pressure in a local environment outside of a sealed treatment environment provided by a dressing. In many cases, the local ambient pressure can also be the atmospheric pressure at which a wound site is located. Alternatively, the pressure can be greater than a hydrostatic pressure associated with a tissue at the wound site.

As used herein, the term “wounds” may include, for example, chronic, acute, traumatic, subacute, closed surgical wounds or dehiscence wounds, partially thick burns, ulcers (such as, diabetic, compressive, or venous insufficiency ulcers), flaps, and grafts. The wound may also include an open abdomen area of a patient.

As used herein, the term “wound site” may include a tissue site, such as, bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or a defect but may be desired to add or promote additional tissue growth. For example, negative pressure therapy can be used in a particular tissue area to grow additional tissue that can be harvested or transplanted to another tissue site. The wound site may also include an area wherein a surgical incision has been previously performed.

Negative pressure wound therapy (NPWT) systems are often used to promote wound healing. In order to heal a wound, the NPWT system applies a negative pressure at a wound site. Since the NPWT system fluidly communicates with the wound site, the NPWT system removes a fluid, i.e., a wound exudate from the wound site by applying the negative pressure on a wound dressing attached about the wound site and collects the fluid in a canister for disposal or analysis. For example, the wound exudate may include slough, necrotic tissue, microbial load (e.g., bacteria and biofilms), etc. The wound exudate removed from the wound site may be collected in a rigid canister for disposal or analysis. Typically, to manage low levels of the exudate, the use of rigid canisters to store the exudate may increase patient discomfort as they may make the NPWT system bulky to handle and may affect a mobility of patient. Further, the rigid canisters may also impact a portability of the NPWT system.

In some cases, conventional wound dressings may include a superabsorbent to absorb the exudate. When such wound dressings get saturated with the exudate, the wound dressings may adversely affect a transfer of the negative pressure to the wound site as the exudate may block pathways for negative pressure flow, which may lead to loss of therapy. In other cases, the exudate collected in the wound dressing itself may present a risk of maceration if the absorbent is fully saturated. Further, some wound dressings may not completely isolate components of the NPWT system, such as, the pump from the exudate, which may allow ingress of the exudate into the components of the NPWT system, which is not desirable. Therefore, there exists a need for a NPWT system that may manage collection of the wound exudate from the wound without disrupting negative pressure therapy at the wound site, without causing skin maceration, and without causing patient discomfort, while improving a portability and an efficacy of the NPWT system.

The present disclosure relates to a flow control device for use with a NPWT system for treatment of a wound site. The fluid control device has a wound dressing, a pump, and an exudate canister configured to receive wound exudate removed from the wound site. The fluid control device includes a first valve disposed in fluid communication with an inlet of the pump. The fluid control device further includes a second valve disposed in fluid communication with an outlet of the pump. The fluid control device further includes a canister assembly. The canister assembly includes a first canister including a first chamber and a first diaphragm disposed in the first chamber and dividing the first chamber into a first air chamber and a first exudate chamber. The first diaphragm further fluidly isolates the first air chamber from the first exudate chamber. The first air chamber is disposed in fluid communication with each of the first valve and the second valve. The first exudate chamber is configured to be in selective fluid communication with the wound dressing and the exudate canister. The canister assembly further includes a second canister fluidly isolated from the first canister. The second canister includes a second chamber and a second diaphragm disposed in the second chamber and dividing the second chamber into a second air chamber and a second exudate chamber. The second diaphragm further fluidly isolates the second air chamber from the second exudate chamber. The second air chamber is disposed in fluid communication with each of the first valve and the second valve. The second exudate chamber is configured to be in selective fluid communication with the wound dressing and the exudate canister. The first valve is configured to selectively fluidly communicate the inlet with one of the first air chamber of the first canister and the second air chamber of the second canister. The second valve is configured to selectively fluidly communicate the outlet with the other of the first air chamber of the first canister and the second air chamber of the second canister.

The fluid control device for use with the NPWT system may allow collection of the wound exudate from the wound site in the exudate canister. The exudate canister may be integrated with the wound dressing, for example, the exudate canister may be mounted to a top layer of the wound dressing, or the exudate canister may be separate and spaced apart from the wound dressing. In some embodiments, the exudate canister may be embodied as a soft canister made of a breathable/deformable material. Thus, the wound dressing may eliminate a need of superabsorbent material within the wound dressing which may otherwise disrupt the negative pressure therapy and cause skin maceration. Further, the fluid control device may eliminate the requirement of a separate rigid canister to store the wound exudate thereby reducing a weight of the NPWT system and improving portability of the NPWT system.

Further, the canister assembly of the fluid control device including the first diaphragm and the second diaphragm may isolate the wound exudate from the pump thereby protecting the pump from contamination or damage that may otherwise be caused by the wound exudate. Thus, the fluid control device may eliminate a need for hydrophobic filters for protecting the pump from any contamination.

The canister assembly of the fluid control device further includes a plurality of fluid passages and a plurality of check valves for directing the wound exudate from the wound dressing to the exudate canister. The fluid passages and the check valves may prevent the exudate canister to be in direct communication with the inlet or the outlet of the pump and thus, may not impede the negative pressure/positive pressure applied by the pump. Further, the canister assembly may prevent the exudate canister to be in direct communication with the wound site, thereby preventing loss of therapy and skin maceration.

Overall, the NPWT system may efficiently manage collection of the wound exudate from the wound without disrupting negative pressure therapy at the wound site, without causing skin maceration, and without causing patient discomfort, while improving a portability and an efficacy of the NPWT system.

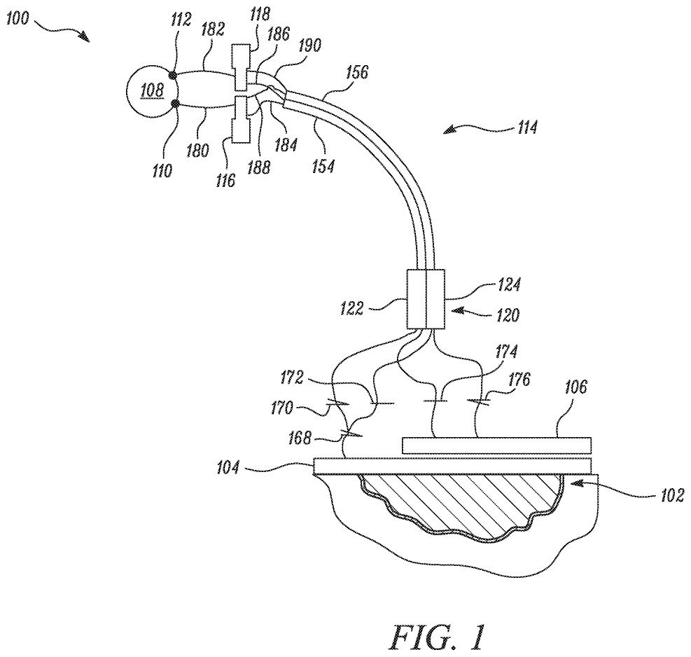

FIG. 1 is a schematic view illustrating a NPWT system 100 for treatment of a wound site 102, according to an embodiment of the present disclosure. The NPWT system 100 includes a wound dressing 104 disposed at the wound site 102. The NPWT system 100 further includes an exudate canister 106 configured to receive wound exudate removed from the wound site 102. The wound exudate may include slough, necrotic tissue, microbial load (e.g., bacteria and biofilms), and the like. Thus, the exudate canister 106 is configured to receive the wound exudate from the wound site 102 which may otherwise harbor bacteria at the wound site 102.

In some embodiments, the exudate canister 106 is integrated with the wound dressing 104. For example, the exudate canister 106 may be disposed on a top layer of the wound dressing 104 as shown in FIG. 1. In other embodiments, the exudate canister 106 may be separate and spaced apart from the wound dressing 104. In some embodiments, the exudate canister 106 is deformable. In some embodiments, the exudate canister 106 may be made of a breathable material. In an example, the exudate canister 106 may include a flexible pouch made of a soft material, such as, but not limited to, a polymer. The exudate canister 106 may be made of, for example, polyurethane. In some examples, at least a portion of the exudate canister 106 may be transparent or semi-transparent, e.g., to permit a visual assessment of the wound exudate being received from the wound site 102.

The NPWT system 100 further includes a pump 108 including an inlet 110 and an outlet 112. The pump 108 is configured to provide a negative pressure at the inlet 110 and a positive pressure at the outlet 112. In some examples, the pump 108 may be a miniature pump or a micropump that may be adapted to maintain adequate therapeutic negative pressure/positive pressure levels. In other examples, the pump 108 may include a diaphragm pump, or any other suitable pump configured to provide the negative pressure at the inlet 110 and the positive pressure at the outlet 112.

The NPWT system 100 further includes a fluid control device 114 for use with the NPWT system 100. The fluid control device 114 includes a first valve 116 disposed in fluid communication with the inlet 110 of the pump 108. The first valve 116 is in fluid communication with the inlet 110 via an inlet fluid line 180. The fluid control device 114 further includes a second valve 118 disposed in fluid communication with the outlet 112 of the pump 108. The second valve 118 is in fluid communication with the outlet 112 via an outlet fluid line 182.

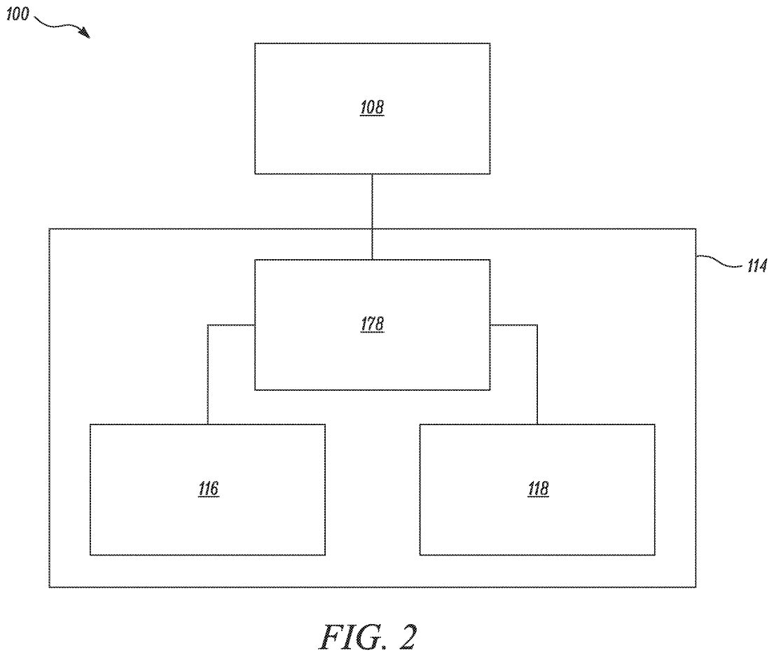

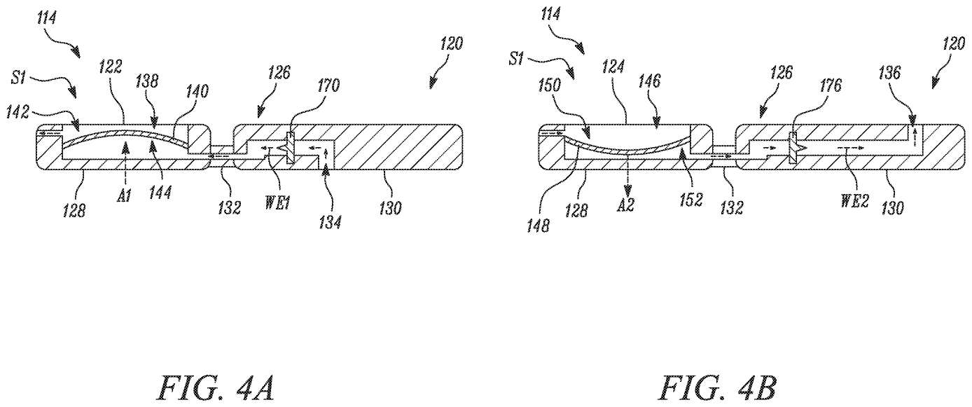

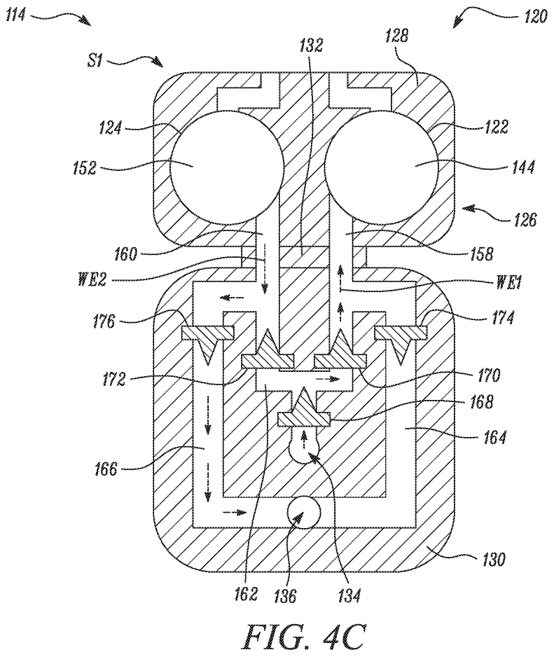

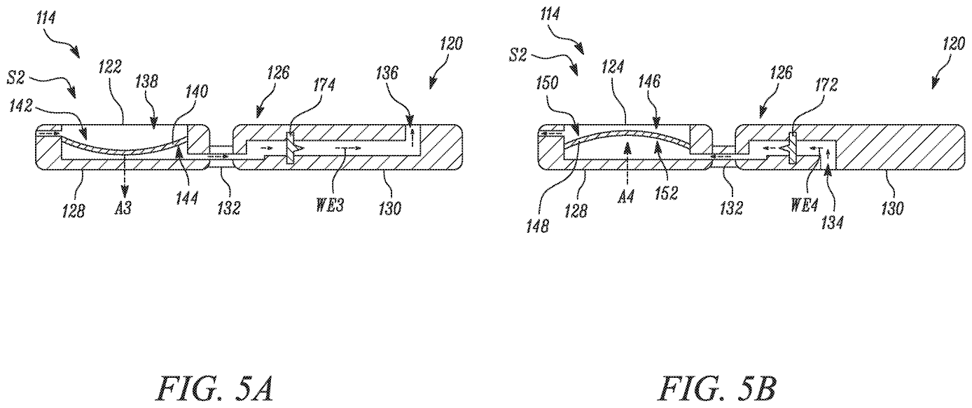

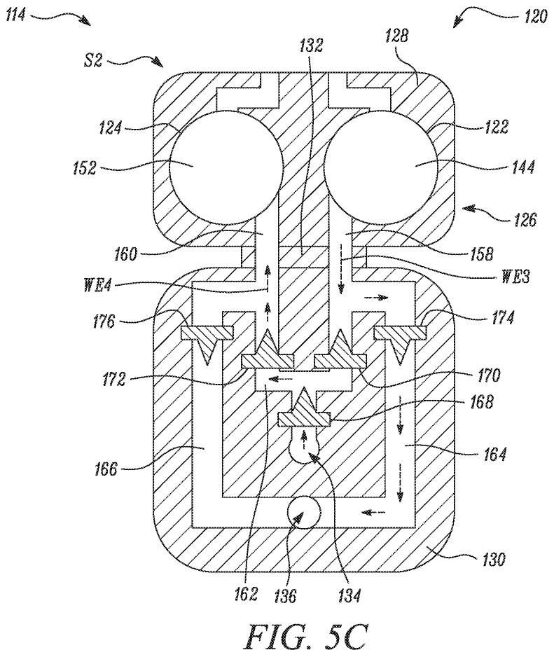

In some embodiments, each of the first valve 116 and the second valve 118 is a solenoid valve. The first valve 116 may receive the negative pressure from the inlet 110 of the pump 108. The second valve 118 may receive the positive pressure from the outlet 112 of the pump 108. Referring now to FIG. 2, a schematic block diagram of the fluid control device 114 of the NPWT system 100 is illustrated. As shown in FIG. 2, the NPWT system 100 further includes a controller 178 communicably coupled to each of the first valve 116 and the second valve 118. The controller 178 is configured to switch the NPWT system 100 between a first state S1 (shown in FIGS. 4A, 4B, and 4C) and a second state S2 (shown in FIGS. 5A, 5B, and 5C). The controller 178 may switch the NPWT system 100 between the first state S1 and the second state S2 after a predefined period of time. For example, the controller 178 may switch the NPWT system 100 between the first state S1 and the second state S2 after every two seconds, or so, without any limitations. The first state S1 and the second state S2 of the NPWT system 100 will be explained in detail later in this section. Further, the controller 178 is also communicably coupled to the pump 108. The controller 178 may adjust an amount of the negative pressure as well as an amount of the positive pressure being applied by the pump 108. In some examples, the controller 178 may control a speed and/or a stroke of the pump 108. In some examples, the controller 178 may control a prime mover (e.g., an electric motor) driving the pump 108. The controller 178 may also control an activation and a deactivation of the pump 108. Further, the controller 178 may control respective electric currents supplied to the first valve 116 and the second valve 118.

The controller 178 may include one or more processors and one or more memories. It should be noted that the one or more processors may embody a single microprocessor or multiple microprocessors for receiving various input signals. Numerous commercially available microprocessors may be configured to perform the functions of the one or more processors. Each processor may further include a general processor, a central processing unit, an application specific integrated circuit (ASIC), a digital signal processor, a field programmable gate array (FPGA), a digital circuit, an analog circuit, a controller, a microcontroller, any other type of processor, or any combination thereof. Each processor may include one or more components that may be operable to execute computer executable instructions or computer code that may be stored and retrieved from the one or more memories.

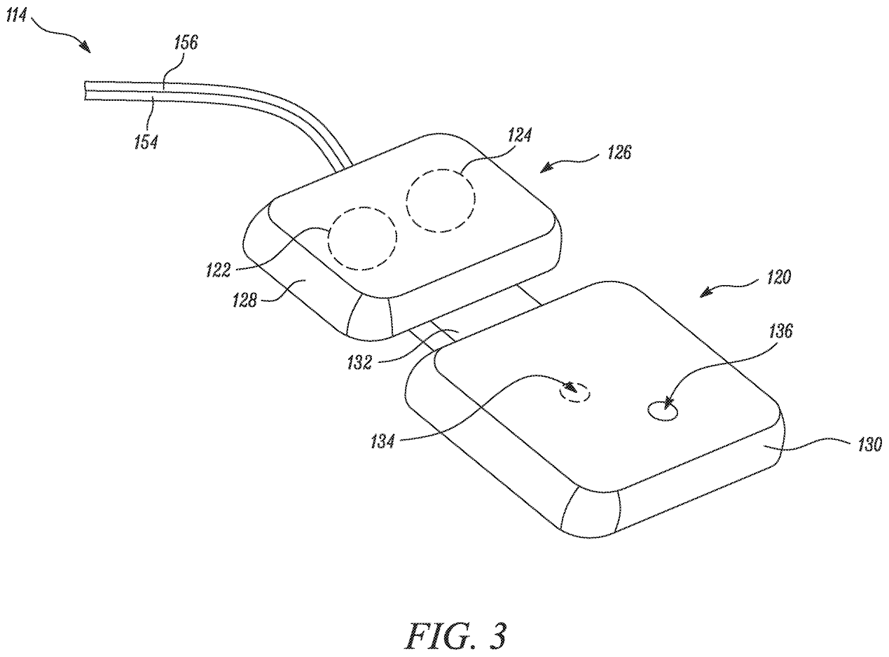

FIG. 3 illustrates a perspective top view of a canister assembly 120. Specifically, the fluid control device 114 includes the canister assembly 120. The canister assembly 120 includes a first canister 122. The canister assembly 120 further includes a second canister 124 fluidly isolated from the first canister 122. In some embodiments, the canister assembly 120 of the fluid control device 114 further includes a housing 126. In some cases, the housing 126 may include a conformable material. In some embodiments, the housing 126 may include a foam.

In some embodiments, the housing 126 includes a first wide section 128, a second wide section 130, and a narrow section 132 connecting the first wide section 128 to the second wide section 130. The housing 126 forms the first canister 122 and the second canister 124. Specifically, the first canister 122 and the second canister 124 are disposed in the first wide section 128 of the housing 126. In the illustrated embodiment of FIG. 3, each of the first wide section 128 and the second wide section 130 is substantially rectangular in shape. In other embodiments, the first wide section 128 and the second wide section 130 may have any other shape, such as, but not limited to circular, square, triangular, and the like.

Further, the housing 126 includes a first opening 134 disposed in fluid communication with the wound dressing 104 (see FIG. 1). Specifically, the first opening 134 is disposed in the second wide section 130 of the housing 126. The housing 126 further includes a second opening 136 spaced apart from the first opening 134. Specifically, the second opening 136 is disposed in the second wide section 130 of the housing 126. The first opening 134 and the second opening 136 are disposed at opposing sides of the housing 126. The second opening 136 is disposed in fluid communication with the exudate canister 106 (see FIG. 1).

As shown in FIG. 4A, the first canister 122 includes a first chamber 138 and a first diaphragm 140 disposed in the first chamber 138. The first diaphragm 140 divides the first chamber 138 into a first air chamber 142 and a first exudate chamber 144. The first diaphragm 140 further fluidly isolates the first air chamber 142 from the first exudate chamber 144. Further, the first exudate chamber 144 is configured to be in selective fluid communication with the wound dressing 104 (see FIG. 1) and the exudate canister 106 (see FIG. 1).

With reference to FIGS. 1 and 4A, the first air chamber 142 is disposed in fluid communication with each of the first valve 116 and the second valve 118. Further, the fluid control device 114 includes a first tube 154 disposed in fluid communication with the first air chamber 142 and configured to be in selective fluid communication with the first valve 116 or the second valve 118. Thus, the first tube 154 establishes fluid communication between the first air chamber 142 and any one of the first valve 116 and the second valve 118. Further, the first valve 116 is in fluid communication with the first tube 154 via a first fluid line 184. Furthermore, the second valve 118 is in fluid communication with the first tube 154 via a second fluid line 186.

As shown in FIG. 4B, the second canister 124 includes a second chamber 146 and a second diaphragm 148 disposed in the second chamber 146. The second diaphragm 148 divides the second chamber 146 into a second air chamber 150 and a second exudate chamber 152. The second diaphragm 148 further fluidly isolates the second air chamber 150 from the second exudate chamber 152. Further, each of the first chamber 138 (see FIG. 4A) and the second chamber 146 is disposed in the first wide section 128 of the housing 126. Furthermore, the second exudate chamber 152 is configured to be in selective fluid communication with the wound dressing 104 (see FIG. 1) and the exudate canister 106 (see FIG. 1).

With reference to FIGS. 1 and 4B, the second air chamber 150 of the second canister 124 is disposed in fluid communication with each of the first valve 116 and the second valve 118. Further, the fluid control device 114 includes a second tube 156 disposed in fluid communication with the second air chamber 150 and configured to be in selective fluid communication with the first valve 116 or the second valve 118. Thus, the second tube 156 establishes fluid communication between the second air chamber 150 and any one of the first valve 116 and the second valve 118. Further, the first valve 116 is in fluid communication with the second tube 156 via a third fluid line 188. Furthermore, the second valve 118 is in fluid communication with the second tube 156 via a fourth fluid line 190.

With reference to FIGS. 1, 4A, and 4B, the first valve 116 is configured to selectively fluidly communicate the inlet 110 of the pump 108 with one of the first air chamber 142 of the first canister 122 and the second air chamber 150 of the second canister 124. Specifically, when the NPWT system 100 is in the first state S1, the first valve 116 is configured to fluidly communicate the inlet 110 of the pump 108 with the first air chamber 142 of the first canister 122. Further, when the NPWT system 100 is in the second state S2, the first valve 116 is configured to fluidly communicate the inlet 110 of the pump 108 with the second air chamber 150 of the second canister 124. The second valve 118 is configured to selectively fluidly communicate the outlet 112 of the pump 108 with the other of the first air chamber 142 of the first canister 122 and the second air chamber 150 of the second canister 124. Specifically, when the NPWT system 100 is in the first state S1, the second valve 118 is configured to fluidly communicate the outlet 112 of the pump 108 with the second air chamber 150 of the second canister 124. Further, when the NPWT system 100 is in the second state S2, the second valve 118 is configured to fluidly communicate the outlet 112 of the pump 108 with the first air chamber 142 of the first canister 122.

The fluid control device 114 for use with the NPWT system 100 may allow collection of the wound exudate from the wound site 102 in the exudate canister 106. As the exudate canister 106 is separate from the wound dressing 104, the fluid control device 114 may eliminate a need of superabsorbent material within the wound dressing 104 which may otherwise disrupt the negative pressure therapy and cause skin maceration. Further, the fluid control device 114 may eliminate the requirement of a separate rigid canister to store the wound exudate thereby reducing a weight of the NPWT system 100 and improving portability of the NPWT system 100.

The canister assembly 120 of the fluid control device 114 including the first diaphragm 140 and the second diaphragm 148 may isolate the wound exudate from the pump 108 thereby protecting the pump 108 from contamination or other damages that may be caused by the wound exudate. Thus, the fluid control device 114 may eliminate any need for hydrophobic filters for protecting the pump 108 from contamination.

FIG. 4C illustrates a sectional bottom view of the canister assembly 120 of FIG. 3 when the NPWT system 100 is in the first state S1. With reference to FIG. 4C, the housing 126 includes a first fluid passage 158 fluidly communicating with the first exudate chamber 144. The housing 126 further includes a second fluid passage 160 fluidly communicating with the second exudate chamber 152. Each of the first fluid passage 158 and the second fluid passage 160 extends from the first wide section 128, through the narrow section 132, and into the second wide section 130.

The housing 126 further includes a fluid junction 162 fluidly communicating the first opening 134 with each of the first fluid passage 158 and the second fluid passage 160. In the illustrated embodiment of FIG. 4C, the fluid junction 162 includes a common passage fluidly communicating with each of the first fluid passage 158 and the second fluid passage 160, and an extension passage fluidly communicating the common passage with the first opening 134. In some examples, the fluid junction 162 may be substantially T-shaped. The housing 126 further includes a third fluid passage 164 fluidly communicating the first fluid passage 158 with the second opening 136. The housing 126 further includes a fourth fluid passage 166 fluidly communicating the second fluid passage 160 with the second opening 136. Each of the fluid junction 162, the third fluid passage 164, and the fourth fluid passage 166 is disposed in the second wide section 130. The third fluid passage 164 fluidly meets the first fluid passage 158 proximal to the narrow section 132 as compared to the fluid junction 162. Similarly, the fourth fluid passage 166 fluidly meets the second fluid passage 160 proximal to the narrow section 132 as compared to the fluid junction 162.

The fluid control device 114 further includes a main check valve 168 disposed in the housing 126 and in fluid communication with the fluid junction 162. Specifically, the main check valve 168 is disposed in the second wide section 130 of the housing 126. The main check valve 168 is configured to provide unidirectional flow from the first opening 134 to the fluid junction 162 to allow flow of the wound exudate from the wound dressing 104 (see FIG. 1) to the fluid junction 162. Further, the main check valve 168 may restrict fluid flow from the fluid junction 162 to the first opening 134. The main check valve 168 may be disposed in fluid communication with the extension passage of the fluid junction 162.

The fluid control device 114 further includes a first check valve 170 disposed in the housing 126 and in fluid communication with the first fluid passage 158 proximal to the fluid junction 162. Specifically, the first check valve 170 is disposed in the second wide section 130 of the housing 126. The first check valve 170 is configured to provide unidirectional flow from the fluid junction 162 to the first fluid passage 158 to allow flow of the wound exudate from the fluid junction 162 to the first exudate chamber 144. Further, the first check valve 170 may restrict fluid flow from the first fluid passage 158 to the fluid junction 162.

The fluid control device 114 further includes a second check valve 172 disposed in the housing 126 and in fluid communication with the second fluid passage 160 proximal to the fluid junction 162. Specifically, the second check valve 172 is disposed in the second wide section 130 of the housing 126. The second check valve 172 is configured to provide unidirectional flow from the fluid junction 162 to the second fluid passage 160 to allow flow of the wound exudate from the fluid junction 162 to the second exudate chamber 152. Further, the second check valve 172 may restrict fluid flow from the second fluid passage 160 to the fluid junction 162.

The fluid control device 114 further includes a third check valve 174 disposed in the housing 126 and in fluid communication with the third fluid passage 164. Specifically, the third check valve 174 is disposed in the second wide section 130 of the housing 126. The third check valve 174 is configured to provide unidirectional flow from the first fluid passage 158 to the second opening 136 to allow flow of the wound exudate from the first exudate chamber 144 to the exudate canister 106 (see FIG. 1). Further, the third check valve 174 may restrict fluid flow from the second opening 136 to the first fluid passage 158.

The fluid control device 114 further includes a fourth check valve 176 disposed in the housing 126 and in fluid communication with the fourth fluid passage 166. Specifically, the fourth check valve 176 is disposed in the second wide section 130 of the housing 126. The fourth check valve 176 is configured to provide unidirectional flow from the second fluid passage 160 to the second opening 136 to allow flow of the wound exudate from the second exudate chamber 152 to the exudate canister 106. Further, the fourth check valve 176 may restrict fluid flow from the second opening 136 to the second fluid passage 160.

In some examples, each of the main check valve 168, the first check valve 170, the second check valve 172, the third check valve 174, and the fourth check valve 176 may include a duckbill check valve. Further, each of the main check valve 168, the first check valve 170, the second check valve 172, the third check valve 174, and the fourth check valve 176 may open due to a pressure differential without any direct intervention by the controller 178 (see FIG. 2).

The fluid junction 162, the first fluid passage 158, the main check valve 168, the third fluid passage 164, the first check valve 170, and the third check valve 174 together allow passage of the wound exudate from the wound site 102 to the exudate canister 106, via the first exudate chamber 144. Further, the fluid junction 162, the second fluid passage 160, the main check valve 168, the fourth fluid passage 166, the second check valve 172, and the fourth check valve 176 together allow passage of the wound exudate from the wound site 102 to the exudate canister 106, via the second exudate chamber 152. The fluid passages (i.e., the fluid junction 162, the first fluid passage 158, the second fluid passage 160, the third fluid passage 164 and the fourth fluid passage 166) and the check valves (i.e., the main check valve 168, the first check valve 170, the second check valve 172, the third check valve 174, and the fourth check valve 176) may prevent the exudate canister 106 to be in direct communication with the inlet 110 (see FIG. 1) or the outlet 112 (see FIG. 1) of the pump 108 (see FIG. 1) and thus, may not impede with the negative pressure/positive pressure being applied by the pump 108. Further, the canister assembly 120 prevents the exudate canister 106 to be in direct communication with the wound site 102, thereby preventing loss of therapy and skin maceration. Overall, the NPWT system 100 may efficiently manage collection of the wound exudate from the wound site 102 without disrupting negative pressure therapy at the wound site 102, without causing skin maceration, and without causing patient discomfort, while improving a portability and an efficacy of the NPWT system 100.

An operation of the NPWT system 100 in the first state S1 will now be explained in relation to FIGS. 1, 2, and 4A to 4C. In the first state S1 of the NPWT system 100, the controller 178 is configured to control the first valve 116 to fluidly communicate the inlet 110 of the pump 108 to the first air chamber 142 of the first canister 122. Specifically, the first valve 116 fluidly communicates the inlet 110 to the first air chamber 142 via the inlet fluid line 180, the first fluid line 184, and the first tube 154. Upon fluid communication of the inlet 110 to the first air chamber 142, the first diaphragm 140 expands the first exudate chamber 144 causing the wound exudate to be drawn from the wound dressing 104 into the first exudate chamber 144. Particularly, upon fluid communication of the inlet 110 to the first air chamber 142, the main check valve 168 and the first check valve 170 open and provide the unidirectional flow of the wound exudate from the first opening 134 to the first exudate chamber 144 via the fluid junction 162 and the first fluid passage 158. It should be noted that the first diaphragm 140 moves in a direction shown by an arrow A1 which causes the first exudate chamber 144 to expand. In the first state S1, the wound exudate flows from the wound dressing 104 towards the first exudate chamber 144 along a path as depicted by arrows WE1.

The controller 178 is further configured to control the second valve 118 to fluidly communicate the outlet 112 of the pump 108 to the second air chamber 150 of the second canister 124. Specifically, the second valve 118 fluidly communicates the outlet 112 to the second air chamber 150 via the outlet fluid line 182, the fourth fluid line 190, and the second tube 156. Upon fluid communication of the outlet 112 to the second air chamber 150, the second diaphragm 148 contracts the second exudate chamber 152 causing the wound exudate to be expelled from the second exudate chamber 152 to the exudate canister 106. Particularly, upon fluid communication of the outlet 112 to the second air chamber 150, the fourth check valve 176 opens and provides the unidirectional flow of the wound exudate from the second exudate chamber 152 towards the exudate canister 106 via the second fluid passage 160, the fourth fluid passage 166 and the second opening 136. It should be noted that the second diaphragm 148 moves in a direction shown by an arrow A2 which causes the second exudate chamber 152 to contract. In the first state S1, the wound exudate flows from the second exudate chamber 152 towards the exudate canister 106 along a path as depicted by arrows WE2.

The operation of the NPWT system 100 in the second state S2 will now be explained in relation to FIGS. 1, 2, and 5A to 5C. In the second state S2 of the NPWT system 100, the controller 178 is configured to control the second valve 118 to fluidly communicate the outlet 112 of the pump 108 to the first air chamber 142 of the first canister 122. Specifically, the second valve 118 fluidly communicates the outlet 112 to the first air chamber 142 via the outlet fluid line 182, the second fluid line 186, and the first tube 154. Upon fluid communication of the outlet 112 to the first air chamber 142, the first diaphragm 140 contracts the first exudate chamber 144 causing the wound exudate to be expelled from the first exudate chamber 144 to the exudate canister 106. Particularly, upon fluid communication of the outlet 112 to the first air chamber 142, the third check valve 174 opens and provides the unidirectional flow of the wound exudate from the first exudate chamber 144 towards the exudate canister 106 via the first fluid passage 158, the third fluid passage 164 and the second opening 136. It should be noted that the first diaphragm 140 moves in a direction shown by an arrow A3 which causes the first exudate chamber 144 to contract. In the second state S2, the wound exudate flows from the first exudate chamber 144 towards the exudate canister 106 along a path as depicted by arrows WE3.

The controller 178 is further configured to control the first valve 116 to fluidly communicate the inlet 110 of the pump 108 to the second air chamber 150 of the second canister 124. Specifically, the first valve 116 fluidly communicates the inlet 110 to the second air chamber 150 via the inlet fluid line 180, the third fluid line 188, and the second tube 156. Upon fluid communication of the inlet 110 to the second air chamber 150, the second diaphragm 148 expands the second exudate chamber 152 causing the wound exudate to be drawn from the wound dressing 104 into the second exudate chamber 152. Particularly, upon fluid communication of the inlet 110 to the second air chamber 150, the main check valve 168 and the second check valve 172 open and provide the unidirectional flow of the wound exudate from the first opening 134 to the second exudate chamber 152 via the fluid junction 162 and the second fluid passage 160. It should be noted that the second diaphragm 148 moves in a direction shown by an arrow A4 which causes the second exudate chamber 152 to expand. In the second state S2, the wound exudate flows from the wound dressing 104 towards the second exudate chamber 152 along a path as depicted by arrows WE4.

Therefore, the first and second valves 116, 118 may allow the negative pressure and the positive pressure generated by the pump 108 at the inlet 110 and the outlet 112, respectively, to actuate the first and second diaphragms 140, 148 in an alternating manner. The NPWT system 100 may also alternate between the first and second states S1, S2 based on the actuation of the first and second diaphragms 140, 148 by the negative pressure and the positive pressure generated by the pump 108. Specifically, in the first state S1, the wound exudate from the wound dressing 104 is received and stored in the first exudate chamber 144, while the wound exudate stored in the second exudate chamber 152 during the previous second state S2 is substantially expelled to the exudate canister 106. Further, in the second state S2, the wound exudate from the wound dressing 104 is received and stored in the second exudate chamber 152, while the wound exudate stored in the first exudate chamber 144 during the previous first state S1 is substantially expelled to the exudate canister 106. The alternating first and second states S1, S2 may therefore allow flow of the wound exudate from the wound dressing 104 to the second exudate chamber 152 without direct fluid communication between the wound dressing 104 and the exudate canister 106.

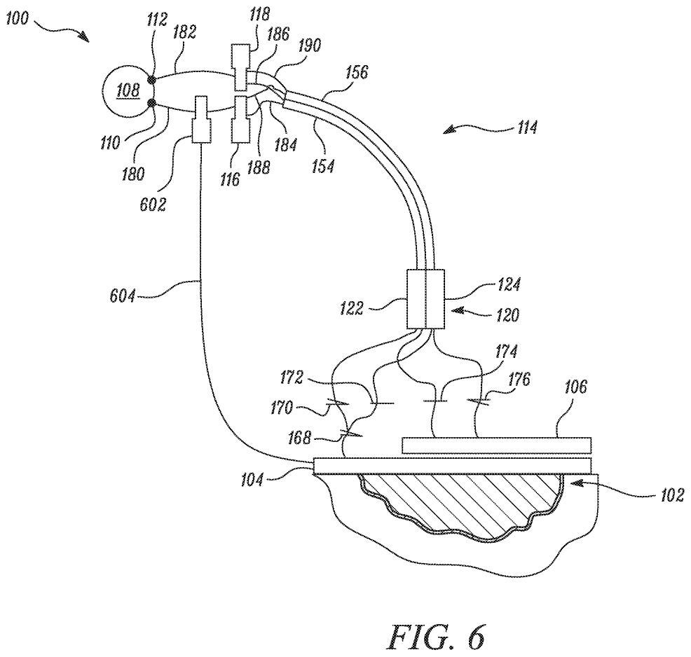

FIG. 6 illustrates the NPWT system 100, according to another embodiment of the present disclosure. The NPWT system 100 shown in FIG. 6 is substantially similar to the NPWT system 100 shown in FIG. 1. However, in the embodiment illustrated on FIG. 6, the NPWT system 100 additionally includes a bypass fluid line 602 connecting the inlet fluid line 180 of the pump 108 with the wound dressing 104. Further, the NPWT system 100 also includes a third valve 604 disposed in the inlet fluid line 180. The third valve 604 is communicably coupled to the controller 178 (see FIG. 2) and may be embodied as a solenoid valve. When the third valve 604 is in a first position, the third valve 604 connects the inlet 110 of the pump 108 with the first valve 116. When the third valve 604 is in a second position, the third valve 604 connects the inlet 110 of the pump 108 with the bypass fluid line 602. In the second position, the third valve 604 allows the negative pressure to be directly applied to the wound dressing 104 via the bypass fluid line 602. The third valve 604 may be disposed in the second position, for example, during initial system priming.

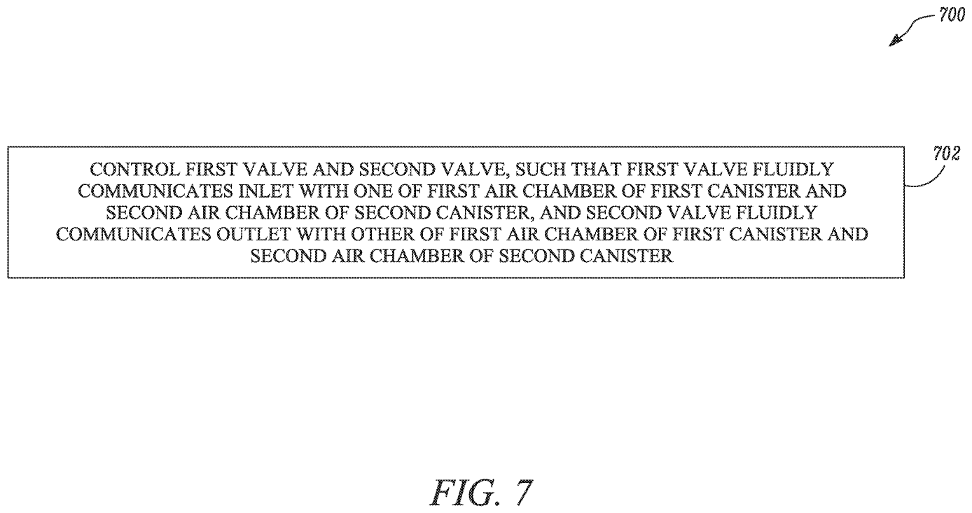

FIG. 7 is a flowchart for a method 700 for controlling fluid flow in the NPWT system 100 of FIG. 1, according to an embodiment of the present disclosure. With reference to FIGS. 1 to 5C and 7, at step 702, the method 700 includes controlling the first valve 116 and the second valve 118, such that the first valve 116 fluidly communicates the inlet 110 with one of the first air chamber 142 of the first canister 122 and the second air chamber 150 of the second canister 124, and the second valve 118 fluidly communicates the outlet 112 with the other of the first air chamber 142 of the first canister 122 and the second air chamber 150 of the second canister 124.

The method 700 further includes controlling, in the first state S1 of the NPWT system 100, the first valve 116 to fluidly communicate the inlet 110 of the pump 108 to the first air chamber 142 of the first canister 122, such that the first diaphragm 140 expands the first exudate chamber 144 causing the wound exudate to be drawn from the wound dressing 104 into the first exudate chamber 144. The method 700 further includes controlling, in the first state S1 of the NPWT system 100, the second valve 118 to fluidly communicate the outlet 112 of the pump 108 to the second air chamber 150 of the second canister 124, such that the second diaphragm 148 contracts the second exudate chamber 152 causing the wound exudate to be expelled from the second exudate chamber 152 to the exudate canister 106.

The method 700 further includes controlling, in the second state S2 of the NPWT system 100, the second valve 118 to fluidly communicate the outlet 112 of the pump 108 to the first air chamber 142 of the first canister 122, such that the first diaphragm 140 contracts the first exudate chamber 144 causing the wound exudate to be expelled from the first exudate chamber 144 to the exudate canister 106. The method 700 further includes controlling, in the second state S2 of the NPWT system 100, the first valve 116 to fluidly communicate the inlet 110 of the pump 108 to the second air chamber 150 of the second canister 124, such that the second diaphragm 148 expands the second exudate chamber 152 causing the wound exudate to be drawn from the wound dressing 104 into the second exudate chamber 152.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below, or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example.

Various examples have been described. These and other examples are within the scope of the following claims.

1. A fluid control device for use with a negative pressure wound therapy (NPWT) system having a wound dressing, a pump, and an exudate canister configured to receive wound exudate removed from a wound site, the fluid control device comprising:
a first valve disposed in fluid communication with an inlet of the pump;
a second valve disposed in fluid communication with an outlet of the pump; and
a canister assembly comprising:
a first canister comprising a first chamber and a first diaphragm disposed in the first chamber and dividing the first chamber into a first air chamber and a first exudate chamber, the first diaphragm further fluidly isolating the first air chamber from the first exudate chamber, wherein the first air chamber is disposed in fluid communication with each of the first valve and the second valve, and wherein the first exudate chamber is configured to be in selective fluid communication with the wound dressing and the exudate canister; and
a second canister fluidly isolated from the first canister, the second canister comprising a second chamber and a second diaphragm disposed in the second chamber and dividing the second chamber into a second air chamber and a second exudate chamber, the second diaphragm further fluidly isolating the second air chamber from the second exudate chamber, wherein the second air chamber is disposed in fluid communication with each of the first valve and the second valve, and wherein the second exudate chamber is configured to be in selective fluid communication with the wound dressing and the exudate canister;
wherein the first valve is configured to selectively fluidly communicate the inlet with one of the first air chamber of the first canister and the second air chamber of the second canister, and wherein the second valve is configured to selectively fluidly communicate the outlet with the other of the first air chamber of the first canister and the second air chamber of the second canister.

2. The fluid control device of claim 1, wherein each of the first valve and the second valve is a solenoid valve.

3. The fluid control device of claim 1, further comprising a controller communicably coupled to each of the first valve and the second valve, wherein the controller is configured to switch the NPWT system between a first state and a second state;
wherein, in the first state of the NPWT system, the controller is configured to:
control the first valve to fluidly communicate the inlet of the pump to the first air chamber of the first canister, wherein, upon fluid communication of the inlet to the first air chamber, the first diaphragm expands the first exudate chamber causing the wound exudate to be drawn from the wound dressing into the first exudate chamber; and
control the second valve to fluidly communicate the outlet of the pump to the second air chamber of the second canister, wherein, upon fluid communication of the outlet to the second air chamber, the second diaphragm contracts the second exudate chamber causing the wound exudate to be expelled from the second exudate chamber to the exudate canister; and
wherein, in the second state of the NPWT system, the controller is further configured to:
control the second valve to fluidly communicate the outlet of the pump to the first air chamber of the first canister, wherein, upon fluid communication of the outlet to the first air chamber, the first diaphragm contracts the first exudate chamber causing the wound exudate to be expelled from the first exudate chamber to the exudate canister; and
control the first valve to fluidly communicate the inlet of the pump to the second air chamber of the second canister, wherein, upon fluid communication of the inlet to the second air chamber, the second diaphragm expands the second exudate chamber causing the wound exudate to be drawn from the wound dressing into the second exudate chamber.

4. The fluid control device of claim 1, further comprising:
a first tube disposed in fluid communication with the first air chamber and configured to be in selective fluid communication with the first valve or the second valve; and
a second tube disposed in fluid communication with the second air chamber and configured to be in selective fluid communication with the first valve or the second valve.

5. The fluid control device of claim 1, further comprising a housing forming the first canister and the second canister.

6. The fluid control device of claim 5, wherein the housing comprises a foam.

7. The fluid control device of claim 5, wherein the housing comprises:
a first opening disposed in fluid communication with the wound dressing;
a second opening spaced apart from the first opening, wherein the second opening is disposed in fluid communication with the exudate canister;
a first fluid passage fluidly communicating with the first exudate chamber;
a second fluid passage fluidly communicating with the second exudate chamber;
a fluid junction fluidly communicating the first opening with each of the first fluid passage and the second fluid passage;
a third fluid passage fluidly communicating the first fluid passage with the second opening; and
a fourth fluid passage fluidly communicating the second fluid passage with the second opening.

8. The fluid control device of claim 7, further comprising:
a main check valve disposed in the housing and in fluid communication with the fluid junction, wherein the main check valve is configured to provide unidirectional flow from the first opening to the fluid junction to allow flow of the wound exudate from the wound dressing to the fluid junction;
a first check valve disposed in the housing and in fluid communication with the first fluid passage proximal to the fluid junction, wherein the first check valve is configured to provide unidirectional flow from the fluid junction to the first fluid passage to allow flow of the wound exudate from the fluid junction to the first exudate chamber; and
a second check valve disposed in the housing and in fluid communication with the second fluid passage proximal to the fluid junction, wherein the second check valve is configured to provide unidirectional flow from the fluid junction to the second fluid passage to allow flow of the wound exudate from the fluid junction to the second exudate chamber.

9. The fluid control device of claim 7, further comprising:
a third check valve disposed in the housing and in fluid communication with the third fluid passage, wherein the third check valve is configured to provide unidirectional flow from the first fluid passage to the second opening to allow flow of the wound exudate from the first exudate chamber to the exudate canister; and
a fourth check valve disposed in the housing and in fluid communication with the fourth fluid passage, wherein the fourth check valve is configured to provide unidirectional flow from the second fluid passage to the second opening to allow flow of the wound exudate from the second exudate chamber to the exudate canister.

10. The fluid control device of claim 7, wherein the housing comprises a first wide section, a second wide section, and a narrow section connecting the first wide section to the second wide section, wherein each of the first chamber and the second chamber is disposed in the first wide section, wherein each of the first fluid passage and the second fluid passage extends from the first wide section, through the narrow section, and into the second wide section, and wherein each of the fluid junction, the third fluid passage, and the fourth fluid passage is disposed in the second wide section.

11. A negative pressure wound therapy (NPWT) system for treatment of a wound site, the NPWT system comprising:
a wound dressing disposed at the wound site;
an exudate canister configured to receive wound exudate removed from the wound site;
a pump comprising an inlet and an outlet, the pump being configured to provide a negative pressure at the inlet and a positive pressure at the outlet;
a first valve disposed in fluid communication with the inlet of the pump;
a second valve disposed in fluid communication with the outlet of the pump; and
a canister assembly comprising:
a first canister comprising a first chamber and a first diaphragm disposed in the first chamber and dividing the first chamber into a first air chamber and a first exudate chamber, the first diaphragm further fluidly isolating the first air chamber from the first exudate chamber, wherein the first air chamber is disposed in fluid communication with each of the first valve and the second valve, and wherein the first exudate chamber is configured to be in selective fluid communication with the wound dressing and the exudate canister; and
a second canister fluidly isolated from the first canister, the second canister comprising a second chamber and a second diaphragm disposed in the second chamber and dividing the second chamber into a second air chamber and a second exudate chamber, the second diaphragm further fluidly isolating the second air chamber from the second exudate chamber, wherein the second air chamber is disposed in fluid communication with each of the first valve and the second valve, and wherein the second exudate chamber is configured to be in selective fluid communication with the wound dressing and the exudate canister;
wherein the first valve is configured to selectively fluidly communicate the inlet with one of the first air chamber of the first canister and the second air chamber of the second canister, and wherein the second valve is configured to selectively fluidly communicate the outlet with the other of the first air chamber of the first canister and the second air chamber of the second canister.

12.-20. (canceled)

21. The NPWT system of claim 11, wherein the exudate canister is integrated with the wound dressing.

22. The NPWT system of claim 11, wherein the exudate canister is separate and spaced apart from the wound dressing.

23. The NPWT system of claim 11, wherein the exudate canister is deformable.

24. A method for controlling fluid flow in the NPWT system of claim 11, the method comprising controlling the first valve and the second valve, such that the first valve fluidly communicates the inlet with one of the first air chamber of the first canister and the second air chamber of the second canister, and the second valve fluidly communicates the outlet with the other of the first air chamber of the first canister and the second air chamber of the second canister.

25. The method of claim 24, further comprising:
controlling, in a first state of the NPWT system, the first valve to fluidly communicate the inlet of the pump to the first air chamber of the first canister, such that the first diaphragm expands the first exudate chamber causing the wound exudate to be drawn from the wound dressing into the first exudate chamber; and
controlling, in the first state of the NPWT system, the second valve to fluidly communicate the outlet of the pump to the second air chamber of the second canister, such that the second diaphragm contracts the second exudate chamber causing the wound exudate to be expelled from the second exudate chamber to the exudate canister.

26. The method of claim 24, further comprising:
controlling, in a second state of the NPWT system, the second valve to fluidly communicate the outlet of the pump to the first air chamber of the first canister, such that the first diaphragm contracts the first exudate chamber causing the wound exudate to be expelled from the first exudate chamber to the exudate canister; and
controlling, in the second state of the NPWT system, the first valve to fluidly communicate the inlet of the pump to the second air chamber of the second canister, such that the second diaphragm expands the second exudate chamber causing the wound exudate to be drawn from the wound dressing into the second exudate chamber.
